(19) United States
(12) Patent Application Publication
(10) Pub. No.: US 2025/0213193 A1
(43) Pub. Date: Jul. 3, 2025

(54) STRETCHABLE ENCAPSULATION FOR IMPLANTABLE BIOMEDICAL DEVICES

(71) Applicant: University of Southern California, Los Angeles, CA (US)

(72) Inventors: Hangbo Zhao, Los Angeles, CA (US); Xinghao Huang, Los Angeles, CA (US)

(21) Appl. No.: 19/004,090

(22) Filed: Dec. 27, 2024

Related U.S. Application Data
(60) Provisional application No. 63/615,744, filed on Dec. 28, 2023.

(51) Int. Cl. A61B 5/00; A61B 5/0538; A61B 5/20

(52) U.S. Cl. CPC A61B 5/686; A61B 5/0538; A61B 5/204; A61B 2562/0214; A61B 2562/0261; A61B 2562/04; A61B 2562/164

(57) ABSTRACT

An implantable device configured to be used within a human body that includes a plurality of electrodes positioned between at least one encapsulation layer and an outer film layer. The outer film layer is configured to flex or stretch and including a biocompatible material.

### CROSS REFERENCE TO RELATED APPLICATIONS

This application claims priority to U.S. Provisional Appl. No. 63/615,744, filed on Dec. 28, 2023, which is herein incorporated by this reference in its entirety.

### STATEMENT AS TO FEDERALLY SPONSORED RESEARCH

This invention was made with Government support under Grant No. U41NS129514, awarded by the National Institutes of Health (NIH). The government has certain rights in the invention.

### FIELD

Disclosed herein are devices, systems, and/or methods relating to implantable medical devices and, more particularly, the fabrication and development of devices, systems, and/or methods specially engineered having stretchable encapsulation to provide long-term functionality and stability in the body fluid environment.

### BACKGROUND

Implantable biomedical devices integrated on organ surfaces can provide critical sensing and modulation functions, such as monitoring various physiological signals (e.g., tissue deformation, temperature, biopotentials, and biomarkers), electrical stimulation, and drug delivery. To achieve long-term functionality and stability in the body fluid environment, conventional implantable devices usually incorporate rigid, hermetic encapsulation materials, such as titanium or ceramics.

However, certain organs and tissues undergo mechanical deformations during physiological activities. For example, the skin, muscle, and nerves, can experience as much as 20% strain during normal postural movements; organs such as bladder can have 30 to 130% surface strain when filling from the empty state to full state; and local strain in the gastrointestinal tract, such as the gastric walls, can reach nearly 100%. Therefore, organ- or tissue-interfaced biomedical implants with rigid encapsulation materials cannot conformally interface with soft tissues and follow their deformations. This mismatch in mechanical stretchability could lead to insufficient bioelectronic interfacing, delamination, or failure of the implants during organ deformation.

Recent development in soft wearable and implantable sensors used for sensing electrocardiogram, electromyography, electrocorticography and strain focuses, focus on high stretchability and flexibility, which allow biomedical implants to be conform to organ surfaces and accommodate organ deformations while maintaining their functions. However, a major challenge lies in chronic applications, where sensors need to achieve large stretchability while ensuring long-term device functionality and stability inside the body fluid environment. Commonly used stretchable encapsulation materials, such as silicone elastomers, have relatively high water permeability, leading to degradation of sensing performance and pose safety risks. In addition, the biocompatibility requirements present additional material challenges to stretchable encapsulation for long-term use.

Accordingly, it is desired to develop a method, system, or device for providing stretchable encapsulation of implantable devices that is capable of providing a desired degree of stretchability while ensuring long-term device functionality and stability inside the body fluid environment. This disclosure resolves these and other issues of the art.

### SUMMARY

In some aspects, an implantable device configured to be used within a human body that includes a plurality of electrodes positioned between at least one encapsulation layer and an outer film layer. The outer film layer is configured to flex or stretch and including a biocompatible material.

In some aspects, the at least one encapsulation layer includes a first elastomeric layer positioned on a first side of the plurality of electrodes and a second elastomeric layer positioned on a second side of the plurality of electrodes opposite the first side.

In some aspects, the at least one encapsulation layer includes a first elastomeric layer positioned between electrode layers of the plurality of electrodes.

In some aspects, the implantable device includes a plurality of clincher tabs positioned on a first lateral end of the implantable device in communication with the plurality of electrodes.

In some aspects, the outer layers include a thickness of approximately 350 μm in thickness.

In some aspects, a dielectric layer of the plurality of electrodes includes a thickness between approximately 150 μm to approximately 200 μm.

In some aspects, the plurality of electrodes includes a plurality of nanotube and an elastomer as a dielectric material.

In some aspects, the outer film layer includes an inorganic material layer sandwiched between an outer layer and an inner layer each different from the inorganic material layer.

In some aspects, the outer film layer includes parylene C.

In some aspects, the outer layer and the inner layer include a material water vapor transmission rate (WVTR) lower than parylene.

In some aspects, the outer film layer includes a wavy or wrinkled surface.

In some aspects, the outer film layer includes a Young's modulus of approximately 2.8 GPa.

In some aspects, the outer film layer is configured to be stretched to up to about 60-80% uniaxial strain without fracturing.

In some aspects, the implantable device includes at least approximately 60% stretchability.

In some aspects, the implantable device includes a normalized water vapor transmission rate per mm thickness of approximately 0.07 g·mm²/m²/day.

In some aspects, the implantable device includes a doubled gauge factor.

In some aspects, an implantable device configured to be used within a human body. An outer film layer is configured to enable a desired degree of flex or stretch during use of the implantable device. The outer film layer includes a material with long-term device functionality and stability inside the body fluid environment.

In some aspects, the outer film layer includes a wavy or wrinkled surface.

In some aspects, the outer film layer includes parylene C.

In some aspects, the outer film layer is configured to be stretched to up to about 60-80% uniaxial strain without fracturing.

In some aspects, the outer film layer includes over about 60% uniaxial stretchability.

In some aspects, the outer film layer encapsulates at least a majority portion of a surface of the implantable device.

In some aspects, the outer film layer includes a biocompatible material, and wherein the implantable device includes at least approximately 60% stretchability.

In some aspects, the implantable device includes a normalized water vapor transmission rates of approximately 0.07 g·mm²/m²/day and/or and a doubled gauge factor.

In some aspects, a method of stretchable encapsulation on a stretchable surface of an implantable device. The method can include subjecting a surface of the implantable device to a level of strain; depositing a layer of a material onto the surface of the implantable device that is subjected to the level of strain, wherein the layer of the material is formed from a biocompatible material; and releasing the level of the strain on the surface of the implantable device to cause the layer of material that is deposited thereon to form a flexible surface of the implantable device configured to flex and/or stretch during use.

In some aspects, the subjecting the surface of the implantable device to the level of strain includes uniaxially pre-stretching the implantable device to a pre-strain of between approximately 30 to approximately 150%.

In some aspects, the depositing the layer of the material onto the surface of the implantable device that is subjected to the level of strain includes conformally coating the layer including a thickness of between approximately 0.5 μm to approximately 10 μm, via chemical vapor deposition.

In some aspects, the releasing the level of the strain on the surface of the implantable device to cause the layer of material that is deposited thereon to form the flexible surface includes releasing a pre-strain of the implantable device to induce local buckling in the outer layer to form microscale wrinkle surfaces.

In some aspects, the method can include annealing the deposited layer beyond its glass transition temperature to increase crystallinity and/or form ordered and folded polymer crystallites.

In some aspects, the method can include annealing the deposited layer beyond its glass transition temperature causing a strain range of the implantable device to increase at least 100%.

In some aspects, the flexible surface includes a wavy or wrinkled surface.

In some aspects, the wavy or wrinkled surface displays over about 60% uniaxial stretchability.

In some aspects, the method can include thermal annealing to control microscale wrinkle formation of the wave or wrinkled surface.

In some aspects, the layer of the material includes parylene C.

In some aspects, the step of depositing is formed by chemical vapor deposition.

In some aspects, during the step of subjecting, the implantable device surface is subjected to a pre-strain of approximately 30-150 percent.

In some aspects, the implantable device is a soft strain sensor including at least approximately 60% stretchability.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A illustrates an example exploded perspective view of a capacitive strain sensors according to an embodiment.

FIG. 1B illustrates an example top view of a capacitive strain sensors according to an embodiment.

FIG. 2 illustrates an example cross-section view of a stretchable encapsulated strain sensor with a sandwich configuration according to an embodiment.

FIG. 3A illustrates an example sensor fabrication and encapsulation process according to an embodiment.

FIG. 3B illustrates an example sensor according to an embodiment.

FIG. 3C illustrates an example cross-section of the stretchable encapsulated sensor with wrinkled encapsulation layer.

FIG. 4A illustrates a graph comparing stress on example strain sensors of this disclosure before and after applying a wrinkle coating according to an embodiment.

FIG. 4B illustrates a graph showing relative capacitance change of an example strain sensor of this disclosure during uniaxial stretching before and after applying the wrinkled outer layer according to an embodiment.

FIG. 4C illustrates a graph showing time-varying capacitance change from an example strain sensor under 30% uniaxial strain before and after applying the wrinkled parylene according to an embodiment.

FIG. 5A illustrates an example sensor fabrication and encapsulation process according to an embodiment.

FIG. 5B illustrates an example sensor according to an embodiment.

FIG. 5C illustrates scanning electron microscope (SEM) images of a cross-section of the example sensor of FIG. 5B.

FIG. 5D illustrates an optical image of example wrinkled surfaces of an example sensor of this disclosure at approximately 0% strain.

FIG. 5E illustrates an optical image of example wrinkled surfaces of an example sensor of this disclosure at approximately strain close to the effective strain range.

FIG. 5F illustrates an optical image of example wrinkled surfaces of an example sensor of this disclosure at strain greater than the effective strain range.

FIG. 6A illustrates a graph showing example strain range and residual strain of strips of example sensors of this disclosure.

FIG. 6B illustrates a graph showing example calculation of effective strain range from stress-strain curves of example sensors of this disclosure.

FIG. 7A illustrates optical images of an annealed sensor (4 μm parylene, 120% pre-strain) under uniaxial stretching at 0%, 30%, and 60% strain.

FIG. 7B illustrates a graph showing example stress-strain curves of example sensors of this disclosure.

FIG. 8 illustrates example profiles of wrinkle surface of example sensors according to example embodiments.

FIG. 9A illustrates a graph showing example period and amplitude of wrinkle surfaces of example sensors of this disclosure.

FIG. 9B illustrates a representative strain sensor with approximate 120% pre-strain released right after coating before annealing, according to one example.

FIG. 9C illustrates a representative strain sensor with approximate 120% pre-strain released right after coating after annealing, according to one example.

FIG. 10A illustrates a graph comparing strain range before and after thermal annealing of example strips of this disclosure.

FIG. 10B shows example strips and demonstrated thermal effects of thermal annealing on example strips of this disclosure.

FIG. 10C shows a graph demonstrating amplitude and period of wrinkle surfaces formed after annealing according to an example.

FIG. 10D shows example strips and demonstrated thermal effects of thermal annealing on example strips of this disclosure.

FIG. 11 illustrates example images of wrinkle surfaces and related effects of thermal annealing according to example embodiments.

FIG. 12A shows example strips and demonstrated thermal effects of thermal annealing on example strips of this disclosure.

FIG. 12B shows optical images of electrode regions of an example sensors under 0%, 25%, and 50% applied strain according to example embodiments.

FIG. 13A is a graph showing compressive strain in the width versus nominal strain percentage according to example embodiments.

FIG. 13B shows cross-section views of optical images of example sensors under 0%, 25%, and 50% applied strain according to example embodiments.

FIG. 14A is a graph showing experimental and analytical results of an encapsulated sensor compared with a non-encapsulated parallel-plate capacitive strain sensor during uniaxial loading and unloading according to example embodiments.

FIG. 14B is a graph showing relative capacitance change of an example representative sensor under a series of step-up strain of 10% to a maximum of 50% strain followed by step-down strain to the initial state according to example embodiments.

FIG. 15A is a graph showing Effective Young's modulus of the unencapsulated sensors and sensors wrinkled surfaces according to example embodiments.

FIG. 15B is a graph showing water vapor transmission rates (WVTR) values of aspects of example sensors of this disclosure wrinkle surfaces at different thicknesses according to example embodiments.

FIG. 16A is a graph comparing normalized WVTR and stretchability between the wrinkled parylene and previously reported stretchable encapsulation materials according to example embodiments.

FIG. 16B is a graph showing relative capacitance change and phase angle of unencapsulated sensors immersed statically in 50.2° C. phosphate-buffered saline (PBS) for 21 days according to example embodiments.

FIG. 16C is a graph showing relative capacitance change and phase angle of encapsulated sensors immersed statically in 50.2° C. phosphate-buffered saline (PBS) for 21 days according to example embodiments.

FIG. 17 includes graphs showing capacitance measurement of an unencapsulated sensor and an encapsulated example sensor during a thermally accelerated aging test according to example embodiments.

FIG. 18A is a graph that shows relative capacitance changes of the encapsulated sensor under 30% strain on day 1, 7, and 14 with insets showing side-view optical images of the example sensor according to example embodiments.

FIG. 18B is a graph that shows relative capacitance changes of the unencapsulated sensor under 30% strain on day 1, 7, and 14 of aging with insets showing side-view optical images according to example embodiments.

FIG. 19A is a graph that shows third-degree polynomial relationship between the volume of a bladder phantom and relative capacitance change (5 cycles) used for calibration and volume prediction according to example embodiments.

FIG. 19B is a graph that shows relative capacitance changes of an example strain sensor on day 1 and day 7 during 7 days of the bladder phantom filling (3 hours) and draining (20 seconds) processes that mimic daily urine accumulation and voiding (200 mL injection, 8 cycles a day) according to example embodiments.

FIG. 20A shows images of ex vivo porcine bladder with an example sutured strain at different volumes according to example embodiments.

FIG. 20B is a graph that shows sensor capacitance change as bladder volume changes according to example embodiments.

FIG. 21 illustrates a flowchart for a method, according to an embodiment.

### DETAILED DESCRIPTION

In some examples, a stretchable encapsulation is disclosed that is biocompatible within the human body and is configured to prevent leaking or exposure of potentially hazardous materials from implanted devices. In some aspects, methods, devices, and systems are disclosed that are specially engineered to provide stretchable encapsulation of an implantable device and do so in a manner that ensures long-term device functionality and stability inside the body fluid environment. In an example, the implantable device is treated or have a coating of a material that is specially formed in a manner that causes the material, e.g., thin film, to have a wavy or wrinkled surface, wherein such wrinkles are provided to enable a desired degree of flex or stretchability when the implantable device is inserted into a human body, and wherein the coating film forming the wrinkled coating is made from a material that is stable inside the body fluid environment.

Prior approaches to stretchable encapsulation for implantable devices have adopted soft elastomers such as polydimethylsiloxane (PDMS) or polyurethane (PU). Despite their mechanical stretchability, their high permeability to gases and vapors can limit the devices' lifetime. Organic and inorganic thin-film materials, such as polyimide, parylene, SU-8, SiO₂, or Al₂O₃ have much improved water vapor transmission rates (WVTR) than elastomers, but they have highly limited intrinsic stretchability (e.g., <1% for inorganic materials and <3% for organic materials). In some aspects, multilayer encapsulation with both stretchable elastomer and non-stretchable thin films can increase the length of diffusion pathway for water molecules, but the stretchability of such composite configuration is limited by the non-stretchable thin films. Wrinkled, wavy thin films could provide a means of achieving both stretchability and encapsulation. Previous research has shown that thin-film SiO₂ arranged in wrinkled form on elastomeric substrates can have both large stretchability (>20%) and maintain good water barrier properties after cyclic stretching. However, thin-film SiO₂ may degrade in body fluid, especially in highly acidic or basic environments.

In some examples, implantable devices are disclosed that incorporate a novel stretchable encapsulation method that uses a coating. In some aspects, conformal deposition of a thin film (e.g., thin film of parylene) is performed on a pre-stretched, elastomer-based device followed by the release of the pre-strain to create wrinkles in the coating. In some aspects, this stretchable encapsulation strategy can be applied to a soft strain sensor, achieving a combination of over 60% stretchability, a normalized WVTR of 0.07 g·mm²/m²/day, and a doubled gauge factor. A thermal annealing step enables controlled microscale wrinkle formation for thick parylene coating and enhanced encapsulation. Benchtop testing and ex vivo strain sensing on a porcine bladder demonstrate accurate and stable strain sensing performance in fluid environments. The high stretchability, biocompatibility, and encapsulation performance, along with the versatility and scalability of the coating method, make this encapsulation strategy potentially useful to a wide range of soft, implantable devices.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise.

In this disclosure, the term “comprising” is synonymous with “including,” “having,” “containing,” or “characterized by.” These terms are inclusive and open-ended and do not exclude additional, unrecited elements or method steps. By using any of these terms, it is meant that at least the named compound, element, particle, or method step is present in the composition or article or method, but does not exclude the presence of other compounds, materials, particles, method steps, even if the other such compounds, material, particles, method steps have the same function as what is named.

In this disclosure, the phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

In this disclosure, the phrase “consisting essentially of” limits the scope of a claim to the specified materials or steps, plus those that do not materially affect the basic and novel characteristic(s) of the claimed subject matter.

In this disclosure, where publications are referenced, the disclosures of these publications in their entireties are hereby incorporated by reference into this application.

In this disclosure, relative terms, such as “about,” “substantially,” or “approximately” are used to indicate a possible variation of ±10% in the stated value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

In an example, a general strategy on providing stretchable encapsulation for implantable biomedical devices is disclosed. In an example, target devices are mechanically stretched uniaxially or biaxially, followed by conformal coating of a biocompatible thin-film encapsulation material (e.g., in the form of parylene or the like) on the prestretched devices. In an example, a preferred encapsulation material is parylene C, which has FDA-approved Class VI biocompatibility. Subsequent release of the prestretch results in the formation of microscale wrinkles in the encapsulation films, and partial or full recovery of the target devices. The wrinkled encapsulation films maintain the biocompatibility and encapsulation properties, while gaining mechanical stretchability due to the wrinkle formation. Specially, stretching of the devices causes smoothing of the wrinkles without fracturing the encapsulation films. In an example, the mechanical stretchability is governed by the type and extent of prestretch, the materials properties of the encapsulation films, and the target device materials. In an example of preliminary results, over 40% uniaxial stretchability and effective encapsulation in fluid environment through cyclic stretching with thermally accelerated aging tests have been achieved.

In some aspects, a wrinkled parylene coating is disclosed for encapsulation of stretchable strain sensors, which are made of carbon nanotube electrodes and silicone elastomer and in a parallel-plate capacitor configuration. Conformal deposition of a parylene thin film on a pre-stretched, elastomer-based strain sensor in some aspects followed by the release of the pre-strain creates wrinkles in the coating. This wrinkled coating can provide over 60% mechanical stretchability and biocompatible encapsulation. The coating can also increase the gauge factor of the strain sensors from 1 to 2 by suppressing the Poisson's effect of the elastomer-based sensor. We investigate the influences of the coating thickness on the sensor performance and demonstrate the long-term stability of these encapsulated strain sensors through cyclic stretching with thermally accelerated aging tests inside saline. Benchtop experiments on bladder phantom and ex vivo studies on a porcine bladder demonstrate the potential for the encapsulated strain sensors for continuous monitoring of bladder volume. The encapsulation approach presented in this work may be applied to other stretchable, implantable devices.

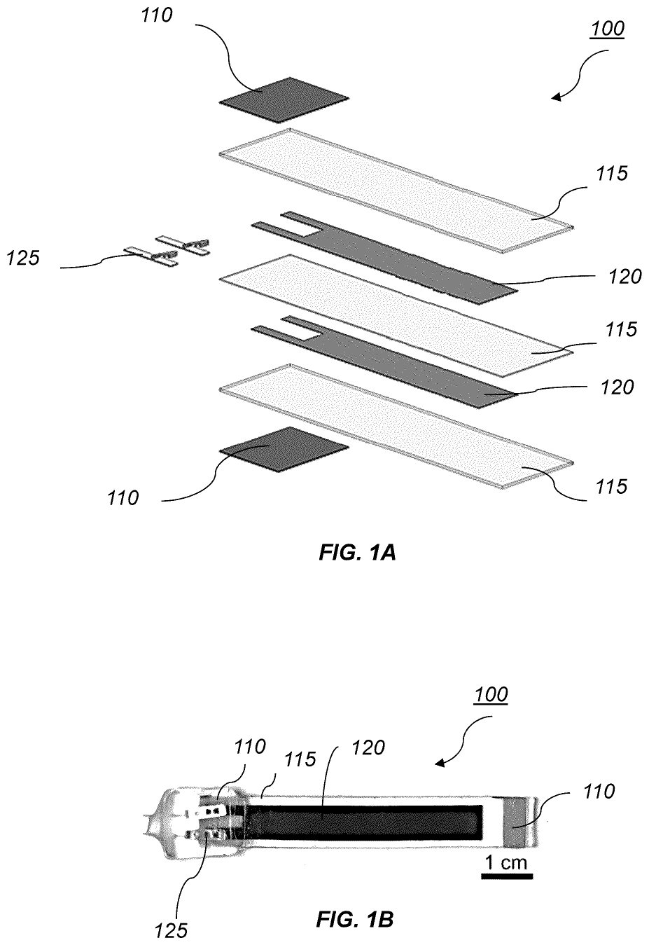

Turning to the figures, FIG. 1A illustrates an example exploded perspective view of a capacitive strain sensor 100 according to an embodiment. In some aspects, sensor 100 can include outer film layers 110 (e.g., one or more parylene film layers). An elastomeric layer 115 (e.g., Ecoflex or any silicone, etc.) and carbon nanotube (CNT) layer 120 can be positioned between layers 110 and at least one layer 115. One or more clincher tabs 125 can be positioned on one end of sensor 100. An example assembled sensor 100 is shown in FIG. 1B.

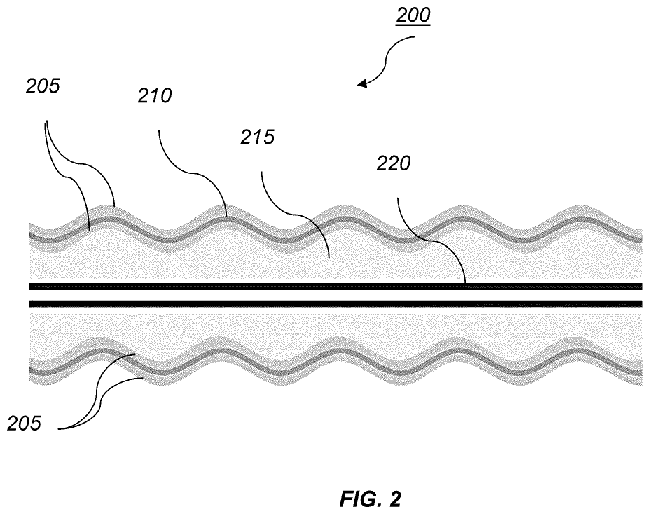

FIG. 2 illustrates an example cross-section view of a stretchable encapsulated strain sensor 200 with a sandwich configuration. In some aspects, sensor 200 can include an inorganic material layer 210 (e.g., a layer of SiO2, Al2O3, etc.) which can include lower WVTR than that of parylene. Layer 210 can be positioned between outer coat layers 205 (e.g., parylene) to form a wrinkled composite barrier layer. A silicone layer 215 and carbon nanotubes (CND) 220 can be positioned between outer layers 205. Positioning layer 210 between layers 205 with equal thickness can advantageously position it at the mechanical neutral plane and reduce bending strain in the inorganic material of layer 210 during wrinkle formation. Inorganic nanoscale thin film materials of layer 210 with ultralow WVTR can be formed using deposition methods such as atomic layer deposition (ALD) or plasma-enhanced chemical vapor deposition (PECVD). The resulting multilayered configuration with alternating organic and inorganic layers can increase the length of diffusion pathways for fluids penetration, leading to significantly improved encapsulation properties. Since the inorganic film of layer 210 serves as the primary water barrier, layer 205 can act as a structural support and protect the inorganic layer 210 from physical damage. Therefore, the thickness of each layer 205 can be reduced to less than 1 μm, which can further lower the stiffness and increases stretchability of the encapsulation layers.

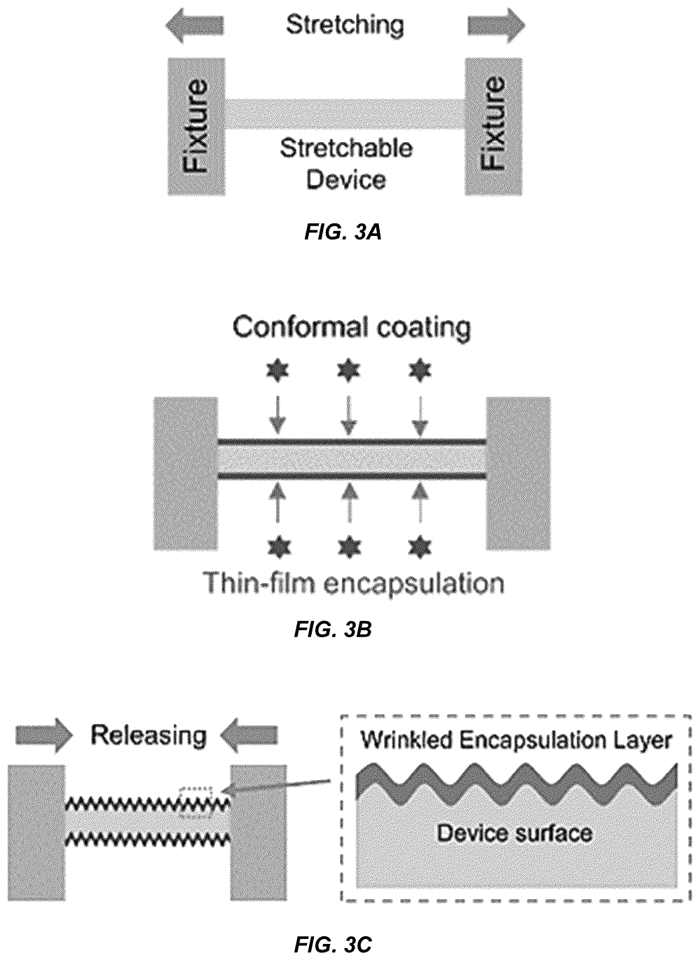

FIGS. 3A to 3C shows steps in an example fabrication process to form wrinkled surfaces of an example sensor (e.g., sensor 100). In the depicted figures, FIG. 3A shows an example stretchable portion of the sensor is pre-stretched by the fixtures at two ends. In FIG. 3B, the pre-stretched device is placed in a coater (e.g., of parylene) to uniformly deposit the outer coat on the device's surface. In FIG. 3C, releasing the pre-stretched device induces local buckling on the outer layer and spontaneously forming wrinkled surfaces (shown in inset). Connecting leads to the sensor and covering the connection with biocompatible epoxy finish the sensor fabrication with a complete encapsulation. FIGS. 3B and 3C show the sensor surface before and after coating.

This disclosure is more clearly understood with corresponding studies discussed more particularly below with respect to aspects, advantages, and fabrication of the herein disclosed strain sensor systems. It is understood that the examples and related data is presented herein for purposes of illustration and should not be construed as limiting the scope of the disclosed technology in any way or excluding any alternative or additional embodiments.

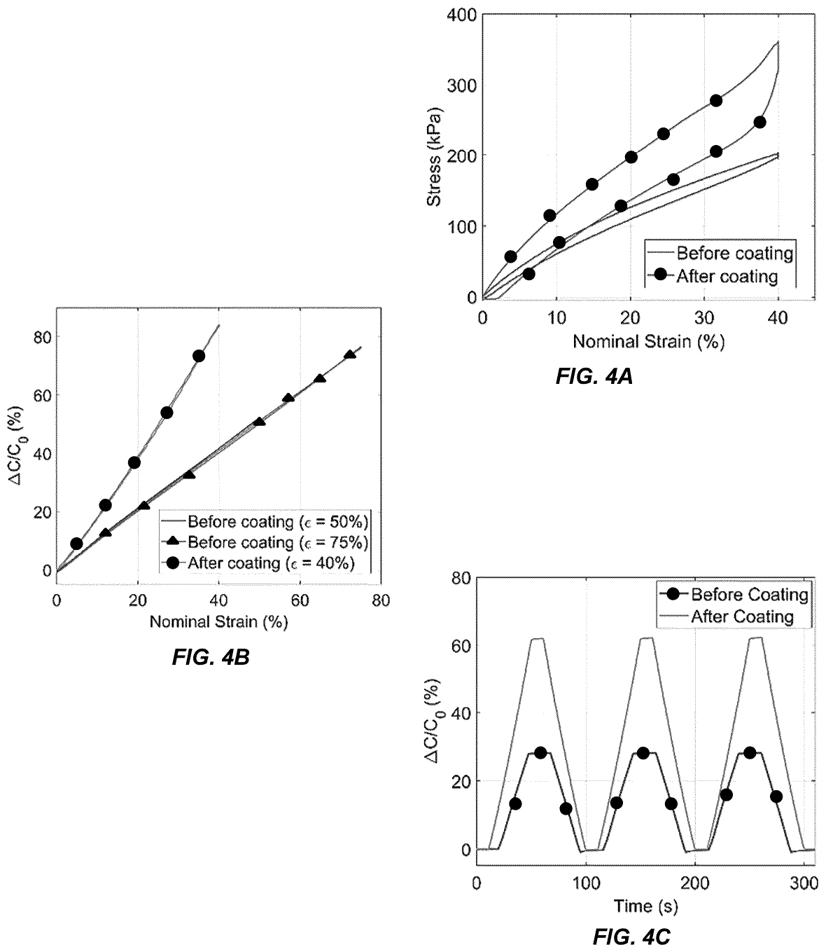

FIGS. 4A and 4B show graphs that demonstrate relative capacitance change of example strain sensors of this disclosure during uniaxial stretching before and after applying the wrinkled parylene. FIG. 4C shows a graph summarizing time-varying capacitance change from an example strain sensor of this disclosure under 30% uniaxial strain before and after applying the wrinkled parylene.

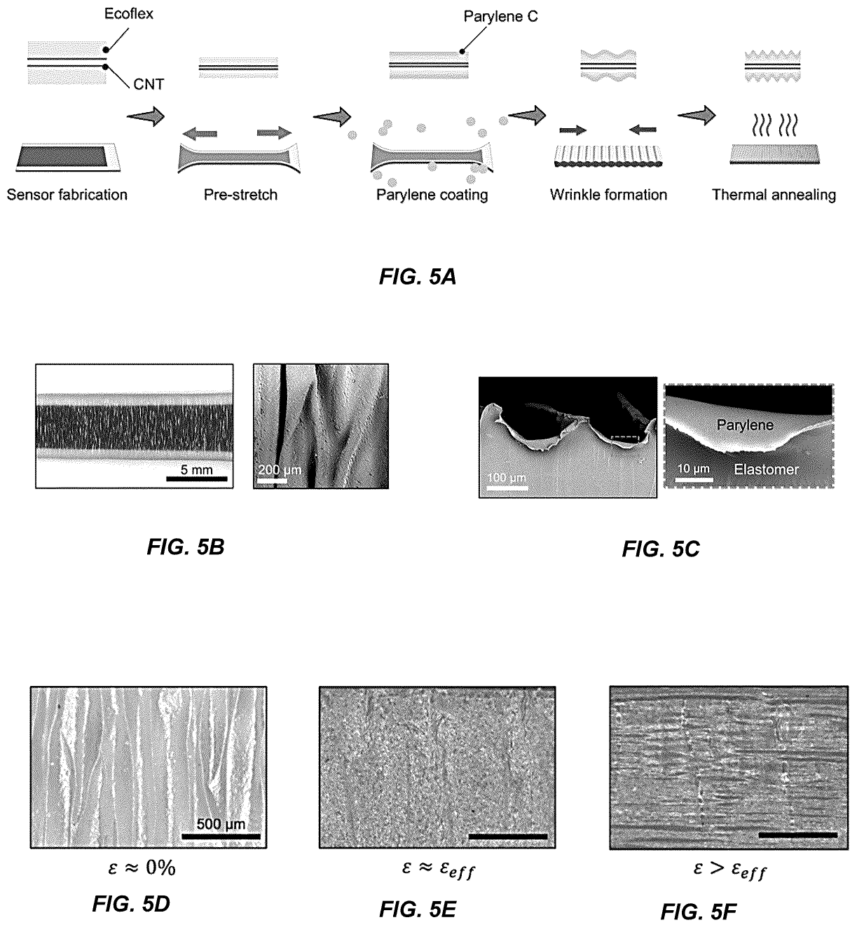

In some aspects, encapsulation of stretchable strain sensors according to this disclosure can commence with uniaxially pre-stretching the sensor to a certain pre-strain (30˜150%) using a custom stretcher (FIG. 5A). The outer layer of FIG. 5A included parylene C as the encapsulation material and a stretchable, parallel-plate capacitive strain sensor as the target device. The strain sensor consists of two stretchable electrodes made of spray-coated multi-walled carbon nanotubes (MWCNTs; 20˜30 μm in thickness) and silicone elastomer (Ecoflex 0031, Young's modulus 68.9 kPa) as the dielectric material (as shown in FIGS. 5A and 5B). The total sensor thickness observed was approximately 750 to 800 μm. Detailed fabrication processes appear in the Materials and Methods. The sensor's capacitance C can be approximately as

$$C = \frac{{ϵ}_{0} {ϵ}_{r} A}{d} ,$$

where ϵ₀ is the permittivity of free space, ϵᵣ is the dielectric constant, A is the overlapping area of the two parallel-plate electrodes, and d is the distance between the two electrodes. Stretching the sensor increases the capacitance due to an increase in A and a decrease in d, with repeatable and stable capacitance change under up to 150% strain.

The pre-stretched sensor can then be conformally coated with a layer of parylene C (thickness: 0.5˜10 μm, Young's modulus ˜2.8 GPa) via a chemical vapor deposition process. Releasing the sensor's pre-strain after coating can induce local buckling in the parylene film, forming microscale wrinkles (FIG. 5B). For relatively thick (>5 μm) parylene coating, a thermal annealing step can increase their stretchability. For example, after parylene coating, releasing can allow wrinkle surfaces to form as well as thermal annealing (e.g., coated sensors annealed in a vacuum oven for 12 hours at 250 C). In some aspects, example sensors of this disclosure can be fabricated using silicone elastomer (Ecoflex 0031, Smooth-On) with part A and B mixed at 1:1 weight ratio. Spin coating can be used to control the thickness of each layer. In some aspects, outer layers of the example sensor can be approximately 350 μm in thickness, while a middle dielectric layer can be approximately 150 to approximately 200 μm thick.

In some aspects, a relatively large pre-strain (e.g., approximately greater than 30%) can lead to the formation of ridges (FIG. 5C) of approximately 3 μm wrinkled with inset showing the elastomer interface (e.g., of parylene/silicone) instead of sinusoidal wrinkles that usually form under small compressive strain. Diffusion of the parylene monomers into the surface layers of porous silicone and polymerization during the coating process results in the formation of a composite layer, confirmed by cross-sectional SEM image of FIG. 5C. This composite interface leads to strong adhesion between the coating and silicone, which is critical to avoid parylene delamination during wrinkle formation and subsequent stretching. FIGS. 5D to 5F show example wrinkle surfaces that are formed by an approximate 2 μm parylene and 90% pre-strain on an elastomer surface without delamination.

The stretchability, or the strain range of sensors of this disclosure, can be determined by the level of pre-strain and outer film thickness (e.g., thickness of parylene layer). To investigate how pre-strain influences the sensor strain range, different pre-strain (30% to 150%) was investigated as to being applied to strips (e.g., elastomeric strip of dimensions: 7.5 mm×50 mm×1 mm) that approximate strain sensors with a parylene thickness of 2 μm. Due to the parylene stiffness, the elastomer strips can become longer (L₁) and cannot recover their initial, unstretched length L₀ after releasing the pre-strain. The resulting residual strain, defined as

$${ε}_{residual} = \frac{{L}_{1}}{{L}_{0}} - 1 ,$$

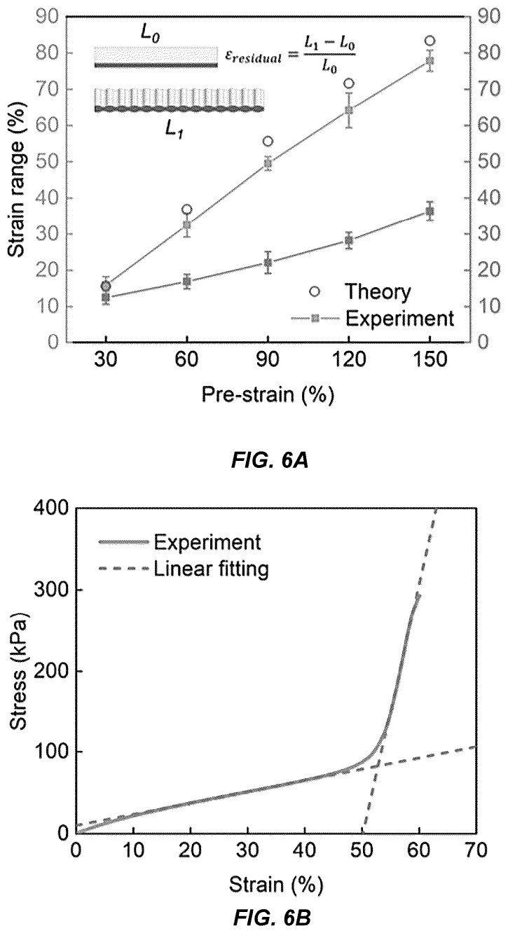

can increase with increasing pre-strain ϵₚᵣₑ (see FIG. 6A). This is likely because strips with more pre-strain have more areas coated by parylene, increasing the resistive force for the strips to shrink back to their original length. The effective strain range ϵ_{eff}, which is the maximum strain to which the elastomer can be stretched without flattening the parylene layer, is then

$${ε}_{eff} = \frac{{ε}_{pre} - {ε}_{residual}}{{ε}_{residual} + 1} .$$

To verify the effective strain range, elastomeric strips with wrinkle surfaces formed by different pre-strain were evaluated. From the experimental stress-strain data illustrated in FIG. 6B, the effective strain range is defined at the strain where the elastomer with wrinkled parylene transits from low modulus to high modulus. In FIG. 6B, the effective strain range is calculated by drawing two linear lines (dashed) tangent to the two linear regions of the stress-strain curve (solid). The intersection point is defined as the effective strain range. Within the effective strain range, the effective modulus of the parylene/silicone/parylene composite is within a range of 140˜240 kPa as the wrinkled parylene coating gradually flattens. Beyond the effective strain range, stretching the flattened parylene coating results in a large increase in the tensile stress, eventually causing fracture in the parylene (see FIGS. 5D to 5F).

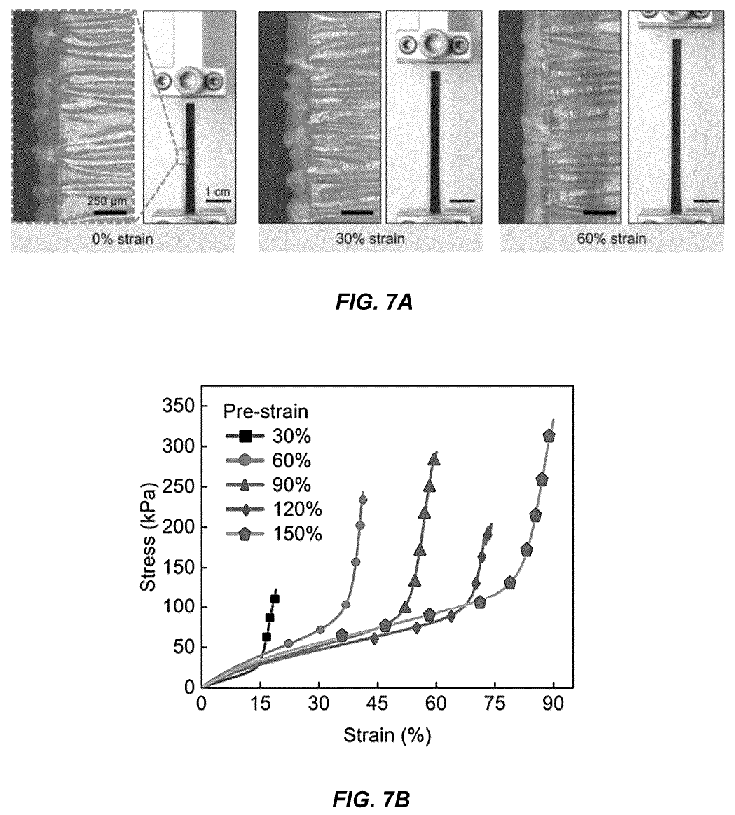

FIG. 7A shows optical images of an example annealed sensor of this disclosure (e.g., 4 μm parylene coating with thermal annealing, 120% pre-strain) under uniaxial stretching at 0%, 30%, and 60% strain demonstrating gradual unfolding of the wrinkles. As shown, the orientation of the wrinkles results in significantly lower stiffness perpendicular to the wrinkles (e.g., length direction) than that parallel to them (width direction). FIG. 7B illustrates a graph showing example stress-strain curves of example elastomer strips of example sensors with approximately 2 μm wrinkled parylene formed at 30%, 60%, 90%, 120%, and 150% pre-strain. FIGS. 7A and 7B demonstrate that larger pre-strain leads to larger strain range and residual strain, and the strain ranges predicted match the experimentally measured values. Stretchability close to 80% is achievable using 150% pre-strain, making the wrinkled parylene encapsulation capable of accommodating large deformations.

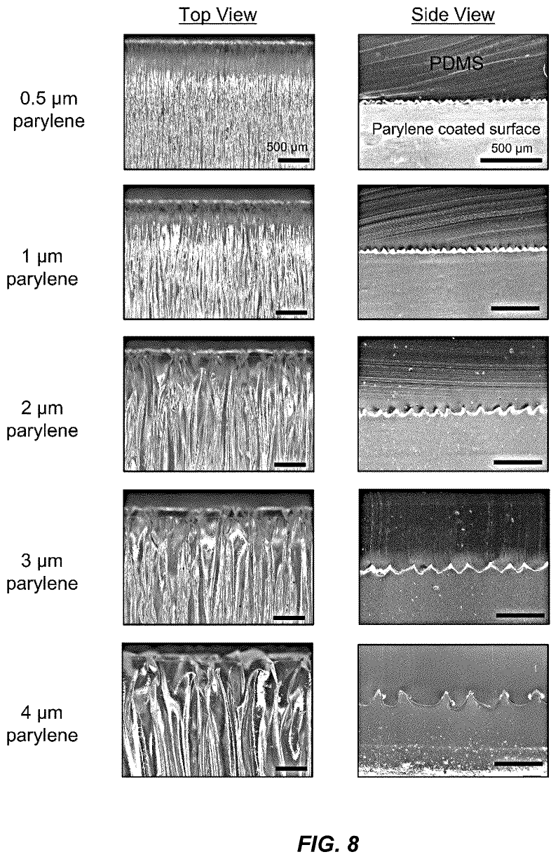

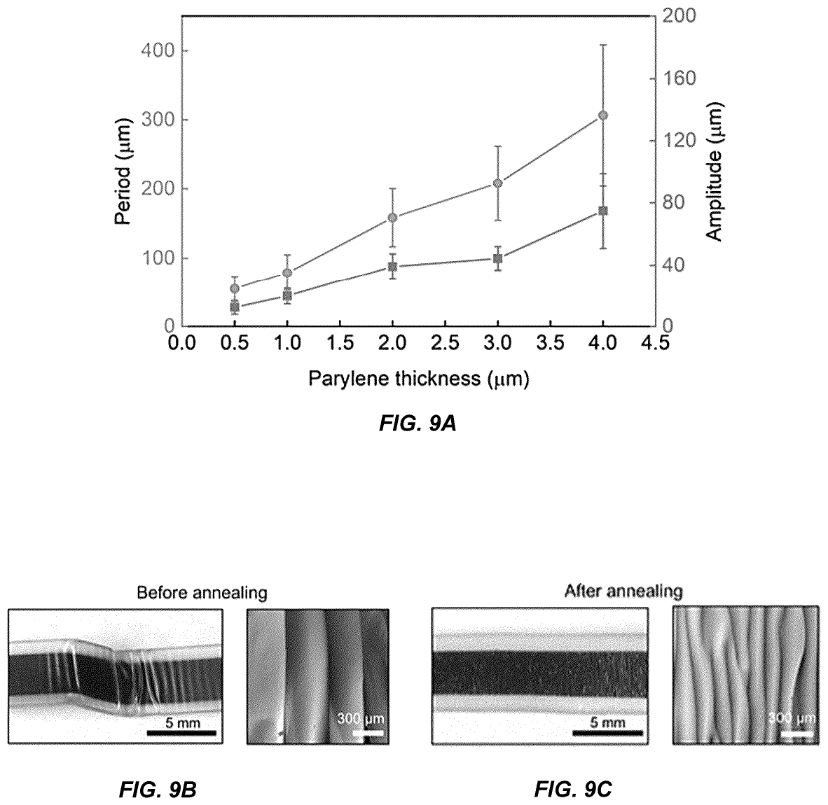

To investigate the effect of parylene thickness on the stretchability, 0.5 to 10 μm-thick parylene was deposited on elastomeric strips pre-stretched to 120%. FIG. 8 illustrates example profiles of wrinkle surface of example sensors according to example embodiments. Specifically, FIG. 8 shows the example profiles of wrinkled surfaces without annealing and formed by approximate 0.5 to 4 μm-thick parylene. The period and amplitude of the wrinkles increase with increasing parylene thickness, as shown in FIG. 9A which shows period and amplitude of wrinkles formed in 0.5 to 4 μm-thick parylene on approximate 1 mm thick Ecoflex strips with 90% pre-strain (n=15 wrinkles for measurements). However, when the parylene thickness is beyond 5 μm, the large thickness ratio between the parylene/silicone/parylene was observed as triggering global buckling of the strips into non-planar structures instead of local wrinkling. FIG. 9B shows a sensor with approximate 6 μm parylene coating after releasing 120% pre-strain. Global buckling was observed as leading to the formation of relatively large wrinkles (e.g., pitch >400 μm) with flat regions on the surfaces, which significantly limit the overall stretchability.

In some aspects, to ensure microscale wrinkle formation for thick parylene coatings, an annealing-assisted wrinkle formation method was investigated. After global buckling, the composite material of the example sensors of this disclosure (e.g., parylene/silicone/parylene composite material) can be annealed at approximately 250° C. under vacuum. The annealing process can result in rearrangement and relaxation of the polymer chains in the semi-crystalline parylene, leading to a decrease in its Young's modulus. The reduction in the parylene layer's stiffness allows the pre-stretched silicone to compress the parylene layer into microscale wrinkles with a period of 170˜200 μm, as illustrated in FIG. 9C. It was observed that thermal annealing can increase parylene crystallinity. Since parylene is a semi-crystalline polymer that contains both amorphous and crystalline regions, annealing parylene beyond its glass transition temperature realigns the entangled and partially ordered polymer chains to form ordered, folded polymer crystallites. Increasing the annealing temperature in some aspects can increase the crystallite size and reduce the spacing between crystallites, and the resulting oxygen and water transmission rates can decrease as the annealing temperature increases. In turn, any encapsulated electronics can have more stable performance and longer lifespans.

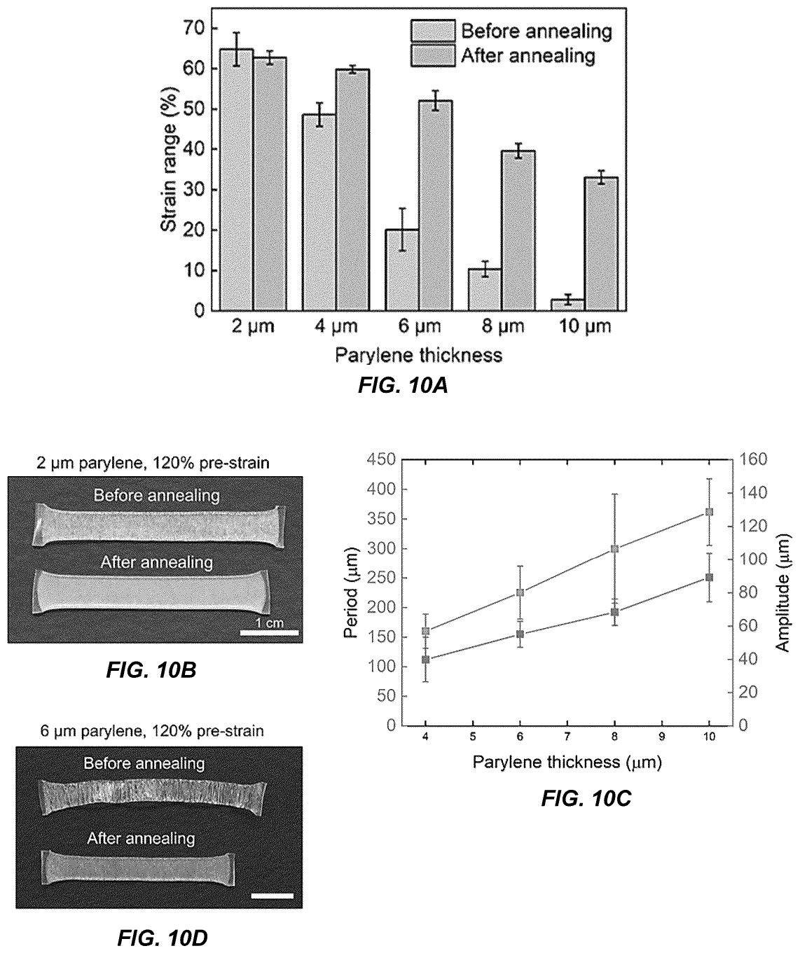

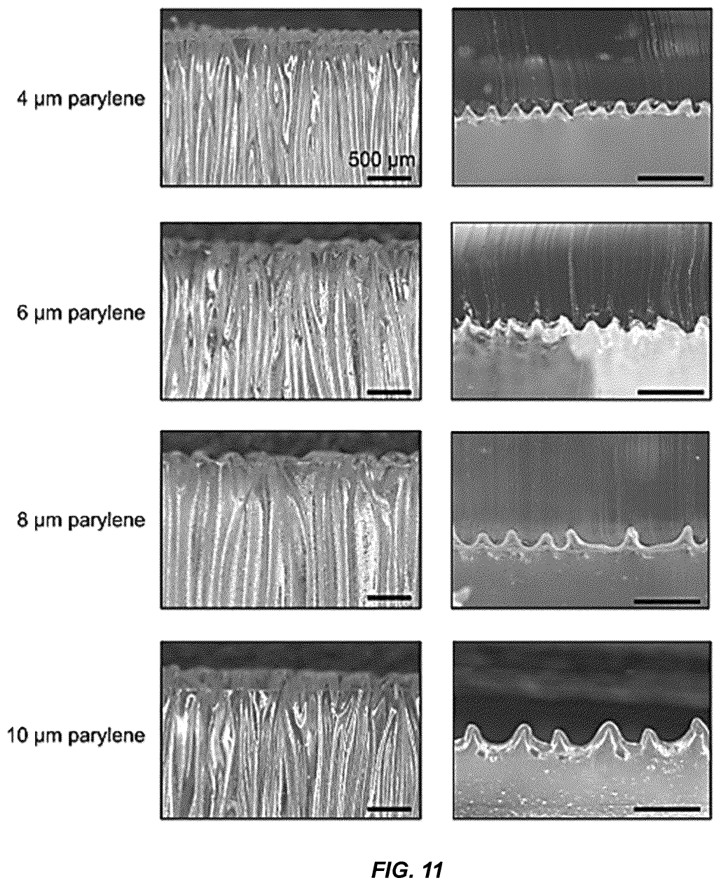

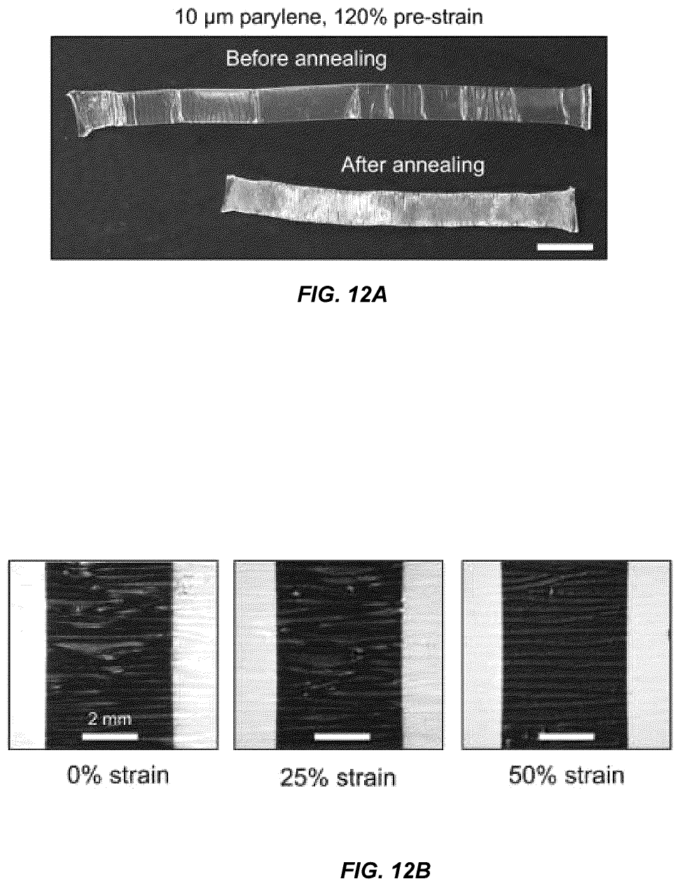

FIG. 10A compares the effective strain ranges before and after annealing of parylene with approximately 2 to approximately 10 μm thicknesses and 120% pre-strain. It was observed that annealing does not significantly change the strain range of the strips with 2 μm parylene since the parylene layer is almost fully compressed, as shown in FIG. 10B. FIG. 10C shows a graph demonstrating amplitude and period of wrinkle surfaces formed after annealing at approximately 4 to approximately 10 μm thick parylene on 1 mm thick Ecoflex strips with 120% pre-strain (n=15 for wrinkle measurement). FIG. 11 shows the wrinkle profiles formed by 4 to 10 μm parylene after annealing. For strips with 4 μm parylene, annealing was observed as reducing the wrinkle period from approximately 300 μm to approximately 160 μm, as shown in FIG. 10C, and residual strain from 28% to 21%, which increases the strain range from 48% to 60%. As the parylene thickness increases, the strain ranges of the strips without annealing decreases sharply, and the strips with 10 μm parylene are almost non-stretchable. FIG. 10D shows that the strip with 6 μm parylene has global bending and non-uniform wrinkle surfaces. Annealing the strip at 250° C. for 3 hours completely transforms its bent shape to flat and the surfaces to densely packed, uniform wrinkles. Depositing 10 μm parylene on pre-stretched strips can barely form wrinkles, yet applying annealing can still transform the flat parylene coating to wrinkles, as shown in FIG. 12A which shows 10 μm wrinkled parylene formed at 120% pre-strain pre-annealing and post-annealing. After annealing, strain range of the strips with 6 μm parylene increases from 20% to 52%, and strain range of the strips with 10 μm parylene increases from 4% to 35%. In some aspects, the annealing process can effectively assist the wrinkle forming for thick parylene and increase the stretchability.

In some aspects, parylene coating and wrinkle surface formation can both provide stretchable encapsulation to capacitive strain sensors as well as cause an increase in their sensitivity (e.g., the gauge factor

$$GF = \frac{Δ C / {C}_{0}}{ε} ) .$$

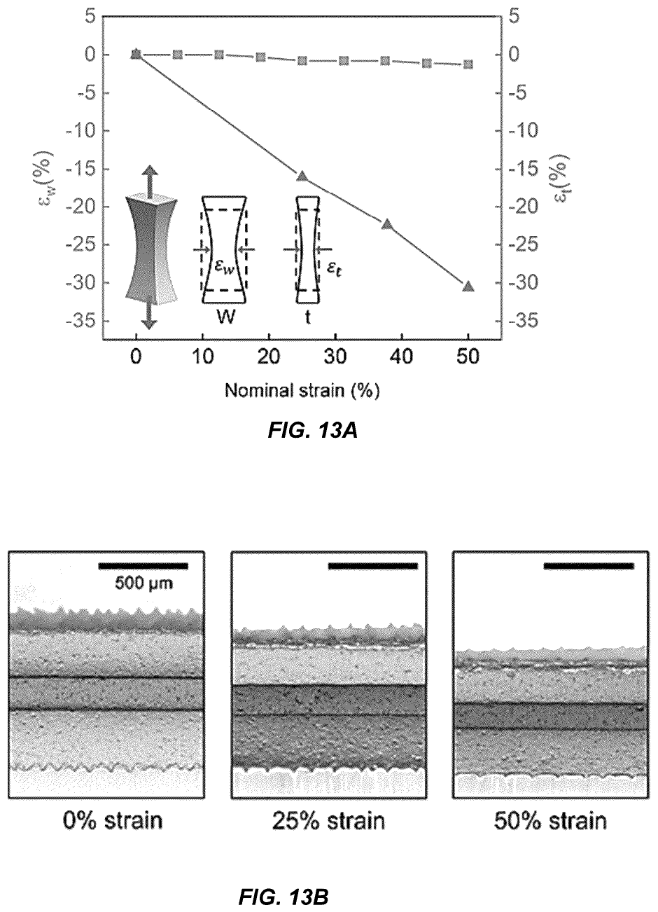

Unlike homogeneous silicone that has a shrinking width during stretching due to Poisson's effect, width of the example sensor with wrinkled parylene has almost no changes, as shown in FIG. 12B which shows optical images of electrode regions of an example sensor. In FIG. 12B, front view optical images of example CNT electrodes region are shown under 0%, 25%, 50% applied strain. In some aspects, given the incompressibility of silicone, small changes in the strain in the width direction ϵ_{w} can lead to large change in the strain in the thickness direction ϵₜ, as shown in FIG. 13A. In some aspects, the observed geometric changes of the parallel-plate capacitor resulted in a capacitance formula for the strain sensor, as follows:

$$C = \frac{{C}_{0} ( 1 + ε )}{( 1 - {v}_{t} ε )} .$$

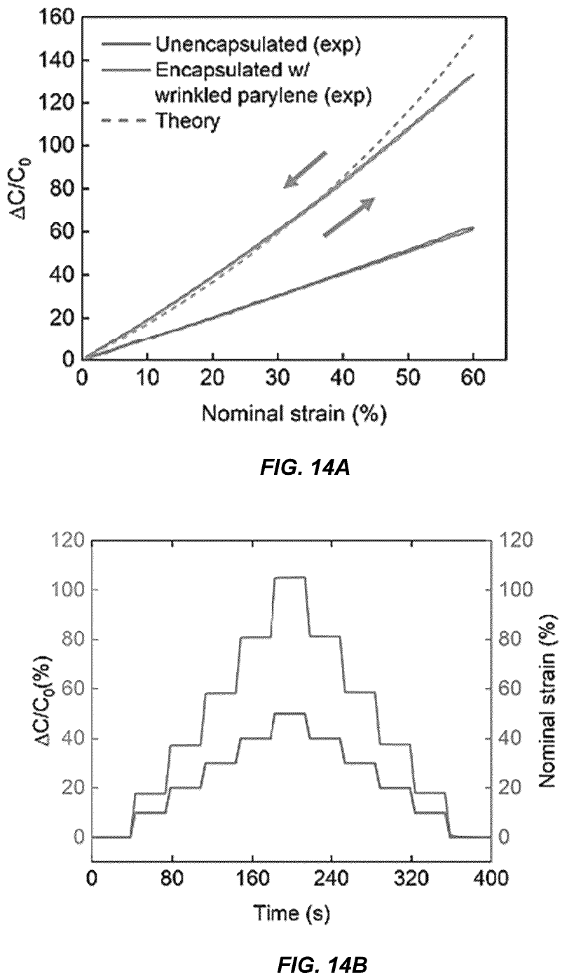

In the above formula, C₀ is the undeformed sensor capacitance, ϵ is the uniaxially applied nominal strain, and vₜ is the Poisson's ratio of the dielectric layer in the thickness direction. Dielectric thickness is measured when the sensor is stretched, as shown in FIG. 13B, and converted to ϵₜ, whose slope yields vₜ=0.61. FIG. 14A shows a graph that compares the measurement of the relative capacitance of encapsulated and unencapsulated sensors when they are stretched to 60% nominal strain. FIG. 14A specifically shows experimental and analytical results of an encapsulated sensor (4 μm parylene, 120% pre-strain) compared with a non-encapsulated parallel-plate capacitive strain sensor during uniaxial loading and unloading under 0-60% strain. The analytical solution that uses the measured vₜ agrees well with the experiment data, and the GF for the encapsulated sensor is 2.18±0.10 (from 5 sensors), in contrast to a GF of 1 for a basic parallel plate capacitive strain sensor.

In some aspects, example sensors with wrinkled surfaces that include parylene encapsulation can exhibit negligible hysteresis and good stability in its capacitance reading. As the capacitance change is caused at least in part by the sensor's geometric change, the hysteresis for the encapsulated sensor is only 1.4% for 60% nominal strain. FIG. 14B is a graph that illustrates relative capacitance change of a representative sensor (4 μm parylene, 120% pre-strain) under a series of step-up strain of 10% to a maximum of 50% strain followed by step-down strain to the initial state and 30 s of holding. The sensor demonstrated no drift in its capacitance reading during both stretching and releasing.

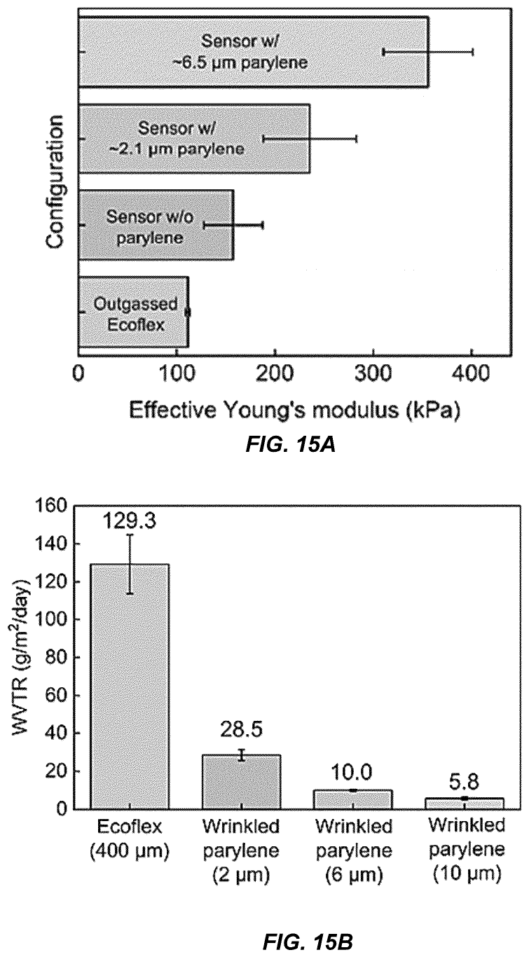

FIG. 15A shows a graph that illustrates Effective Young's modulus of the outgassed Ecoflex, unencapsulated sensors, and sensors with average 2.1 μm and 6.5 μm wrinkled parylene (measured within 35% strain, n=5). FIG. 15A specifically shows the effective Young's moduli of the silicone strips, unencapsulated sensors, sensors with 2˜3 μm of wrinkled parylene (without annealing), and sensors with 6˜7 μm annealed parylene. The sensor's effective Young's modulus only increases from 157 kPa to 235 kPa after forming 2˜3 μm wrinkled parylene, which is smaller than the elastic modulus of human bladder (250 kPa). For thicker parylene coatings, a sensor with 5˜6 μm of wrinkled parylene with annealing has an average effective Young's modulus of 356 kPa. The level of composite material compliance and small sensor thickness (750˜800 μm) contributes to small tensile and bending stiffnesses.

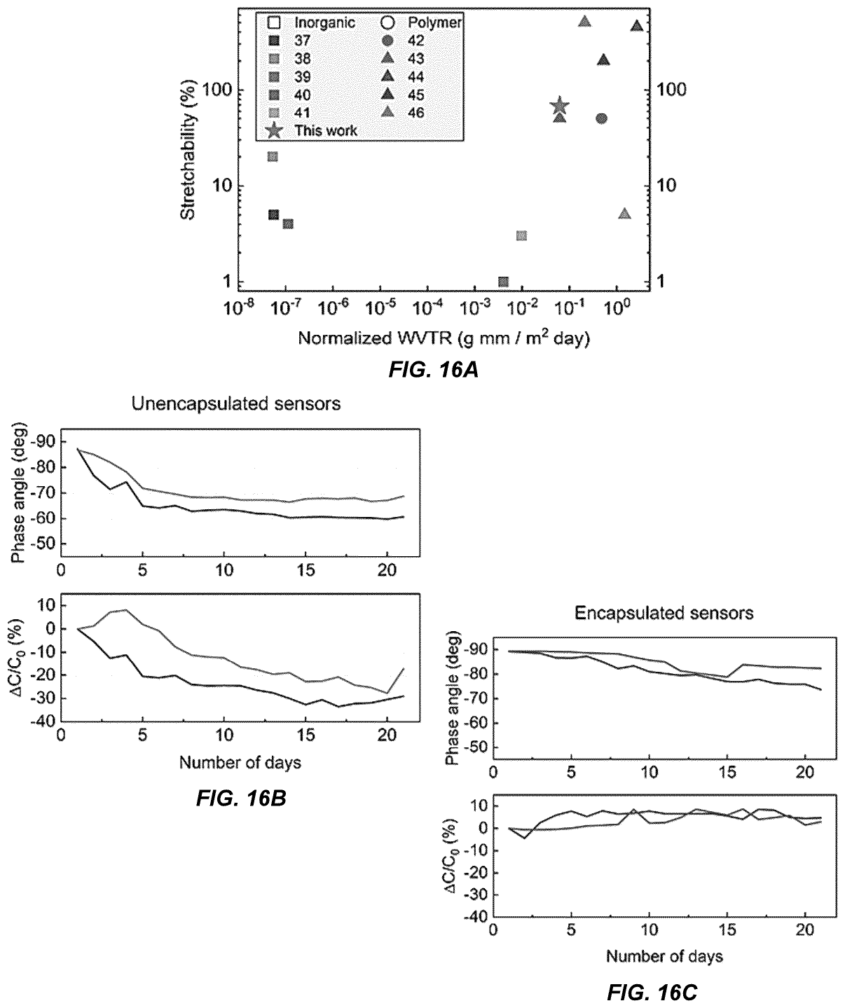

In some aspects, the encapsulation performance of the wrinkled surface of investigated sensors was evaluated through WVTR measurements and thermally accelerated aging tests. WVTR of bare Ecoflex and encapsulated Ecoflex with wrinkled parylene was measured where the bare Ecoflex layer was approximately 400 μm thick, which represents a distance from the center of the example sensor to its surface. The encapsulated Ecoflex (800 μm thick) included 2, 6 and 10 μm-thick wrinkled parylene formed by 120% pre-strain, and the resulting WVTR was multiplied by 2 to represent the rate for one side of the sample. FIG. 15B shows the WVTR values of different combinations of encapsulation layers, including Ecoflex films (approximately 350 to approximately 400 μm) with parylene wrinkles (2, 4, and 6 μm thick, 120% pre-strain) (n=3). The WVTR of pure Ecoflex (129.3 g/m²/day) was observed as more than 4 times of that of the Ecoflex coated by 2 μm wrinkled parylene (28.5 g/m² day). The WVTR of the wrinkled 2 μm parylene on Ecoflex (120% pre-strain) was more than twice of the WVTR of flat plat parylene (13.5 g/m²/day). It was observed that increasing parylene thickness to 6 μm reduces the WVTR to 10.0 g/m²/day, and further increase in the parylene thickness to 10 μm leads to a WVTR of 5.8 g/m²/day. The WVTR of the parylene layer alone in the multilayer system was calculated using the following equation:

$$\frac{1}{{T}_{total}} = {\sum }_{i = 1}^{N} \frac{1}{{T}_{i}}$$

where Tₜₒₜₐₗ was the measured WVTR and Ti was the WVTR of each layer. The WVTR values of 2, 6, 10 μm wrinkled parylene were calculated as 31.5, 10.9, and 5.7 g/m²/day, respectively. The average normalized WVTR (per mm thickness) was then 0.07 g·mm²/m²/day. Comparing the normalized WVTR with other stretchable encapsulation material, as in FIG. 16A, wrinkle surfaces of example sensors of this disclosure with the parylene configuration demonstrated the lowest WVTR among the existing polymer-based stretchable encapsulation layers, while still maintaining stretchability higher than that of the stretchable encapsulation layers made of inorganic materials (strain range≤20%). To achieve large stretchability and optimal encapsulation performance, approximately 4˜6 μm wrinkled parylene with 120% pre-strain was chosen for our implantable strain sensors.

In this example, measurements of baseline capacitances of strain sensors soaked in PBS with and without encapsulation to permit direct comparison of the encapsulation performance. In a static immersion test, an unencapsulated sensors and an encapsulated sensors (120% pre-strain, 6 μm parylene) were completely immersed in PBS at 50.2° C., which corresponded to 2.5 times acceleration compared to the body temperature (37° C.). FIG. 16B shows an unencapsulated sensor's relative capacitance changes and phase angles in 21 days while FIG. 16C shows an encapsulated sensor's relative capacitance changes and phase angles in 21 days. A 30% decrease in the baseline capacitance and a phase angle change from −87.2° to −60° of the non-encapsulated sensor demonstrated charge leakage in the capacitor due to fluid/ion penetration. In contrast, change of baseline capacitance for the encapsulated sensor was maintained within +9% and −1%, with a phase angle sensor of −75° after 3 weeks.

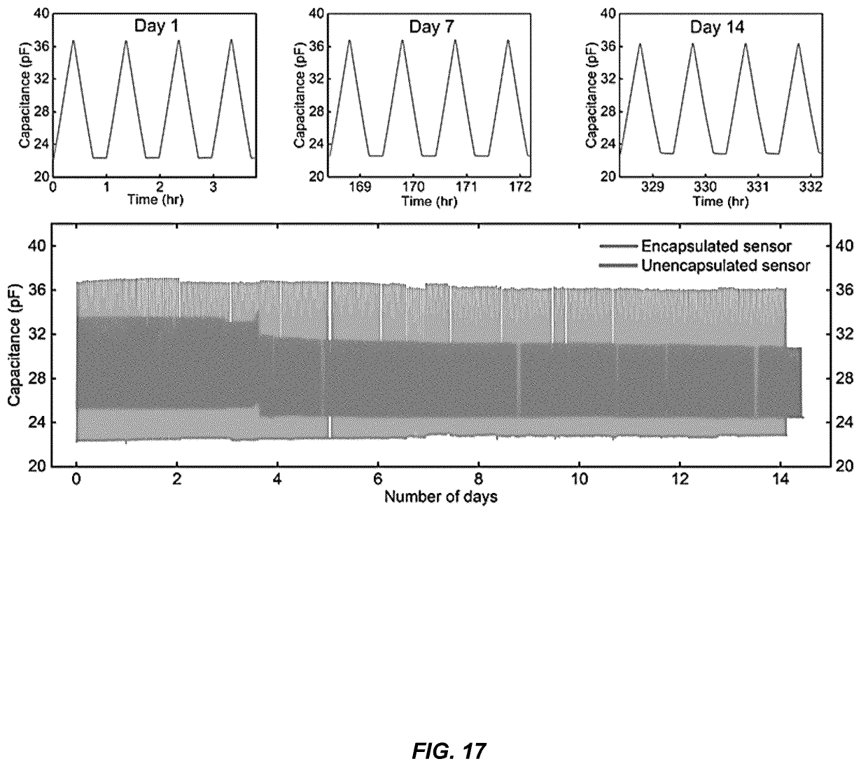

To evaluate the durability and reliability of encapsulated sensors under prolonged mechanical and thermal stress, a thermally accelerated aging test with cyclic stretching was conducted. The encapsulated and unencapsulated sensor were fully immersed in 50.2° C. PBS, with 30% cyclic strain applied to them at a strain rate of 1.35%/min. The capacitance measurement of the encapsulated sensor in FIG. 17, which shows capacitance measurement of an unencapsulated sensor and an example encapsulated sensor (6 μm parylene, 120% pre-strain) stretched by 30% strain within 50.2° C. PBS for 14 days. FIG. 17 demonstrates stable sensing performance for 14 days for the example encapsulated sensor, whereas the unencapsulated sensor has a sudden capacitance decrease after day 3, which is likely caused by charge leakage.

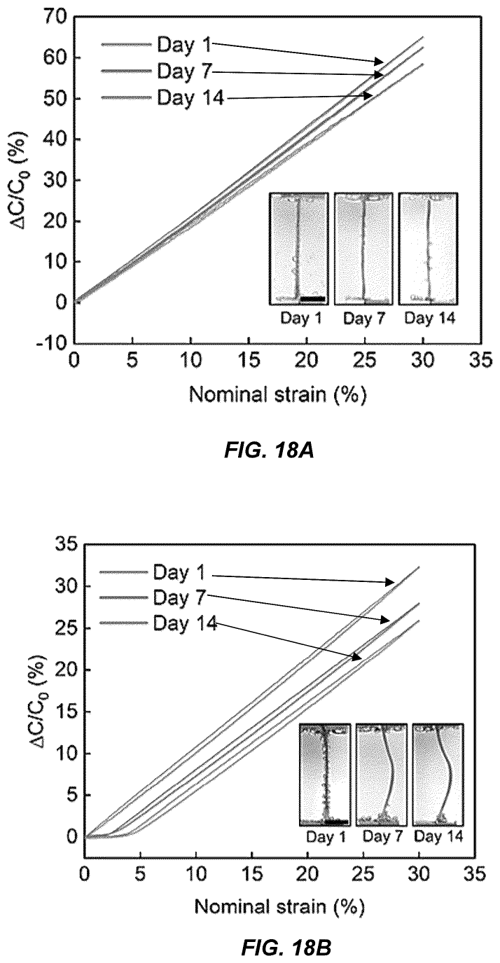

FIG. 18A is a graph that shows the relative capacitance changes with applied strain of the encapsulated sensor before and after 2 weeks of aging with cyclic stretching. In the first week, the GF only decreases by 3.7% (from GF=2.14 to GF=2.06), with no visible changes in the sensor's shape. The GF then decreases by 10.3% (GF=1.93) after 2 weeks, possibly because of the creep in the wrinkled parylene after heating and cyclic stretching (˜1.5% permanent elongation). FIG. 18B shows that the unencapsulated sensor is curved and bent after only 1 week, likely due to swelling of the silicone upon fluid penetration. Swelling increases the length between the two grippers, making the sensor insensitive to small strain (0˜4%) with the gauge factor decreased by 17.0% in 1 week and by 24.9% in 2 weeks. The results demonstrate that the wrinkled parylene encapsulation contributes to stable strain sensing performance.

In some aspects, human bladder was selected as the target model to demonstrate one example use of the encapsulated strain sensors as implantable devices for organ monitoring. Variations in bladder volume and pressure are closely related to its functional states, making these parameters key indicators of urinary health. Abnormal changes in these metrics can signal urinary impairments, such as overactive (OAB) and underactive (UAB) bladder, loss of bladder sensation due to spinal cord injury, or the absence of part of the bladder or sphincter following surgeries. Attaching implantable strain sensors on the bladder enables continuous monitoring of surface strain, which directly correlates to changes in the bladder volume. This capability can provide valuable information for disease diagnosis, support closed-loop neuromodulation, and monitor postoperative recovery.

Since human bladder volume can change from less than 100 mL post-void residual volume to a full capacity of 300-400 mL, high stretchability and durability are required for implantable strain sensors to follow the bladder deformation accurately. To evaluate the sensor functionality in measuring bladder volume, a bladder phantom (60×66 mm ellipsoid, 4 mm wall thickness) was fabricated using silicone (Dragon Skin 15), which has a similar elastic modulus (E=241 kPa within 100% strain) to that of a swine or human bladder (250˜260 kPa). An encapsulated capacitive strain sensor according to one example of this disclosure (6 μm parylene, 120% pre-strain) was sutured on the bladder phantom when the initial water volume within was approximately 100 mL. The bladder phantom is then immersed in PBS at 37° C. to mimic a physiological body fluid environment.

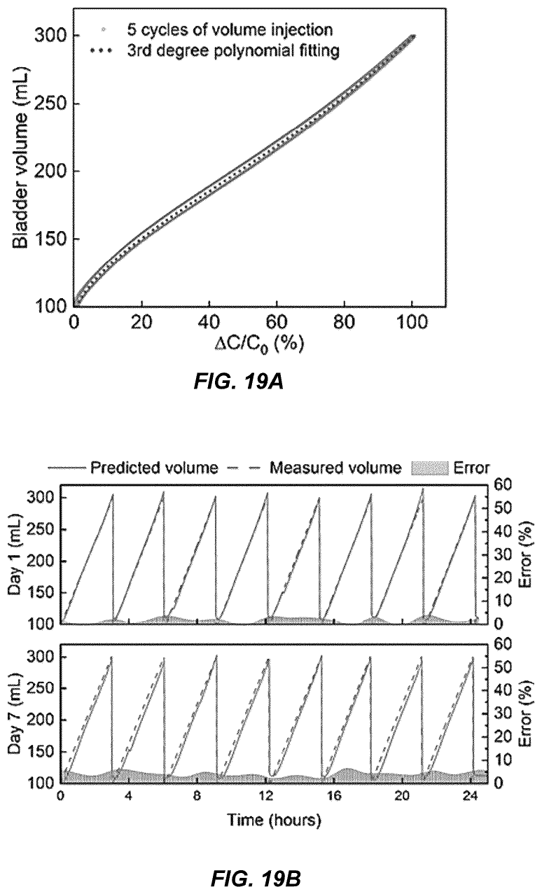

FIG. 19A shows a graph demonstrating that the sensor capacitance changes consistently with water injection, and their relationship can be approximated by a 3rd-degree polynomial fit according to: V=1.5×10⁻⁴C³−0.0233C²+2.8143C+101.6706, where V is bladder volume V(ml) and capacitance change C (%) below. Gradual injection of water (200 mL) into the phantom every 3 hours and quick (e.g., 20s) withdraw after reaching 300 mL mimic the daily urine accumulation and voiding processes. FIG. 19B shows the predicted bladder volume and the actual bladder volume controlled by the syringe pump. During the test, the average relative error is 1.82% on day 1 and 4.24% on day 7, which demonstrates that the example sensor of this disclosure can reliably predict bladder volume over time.

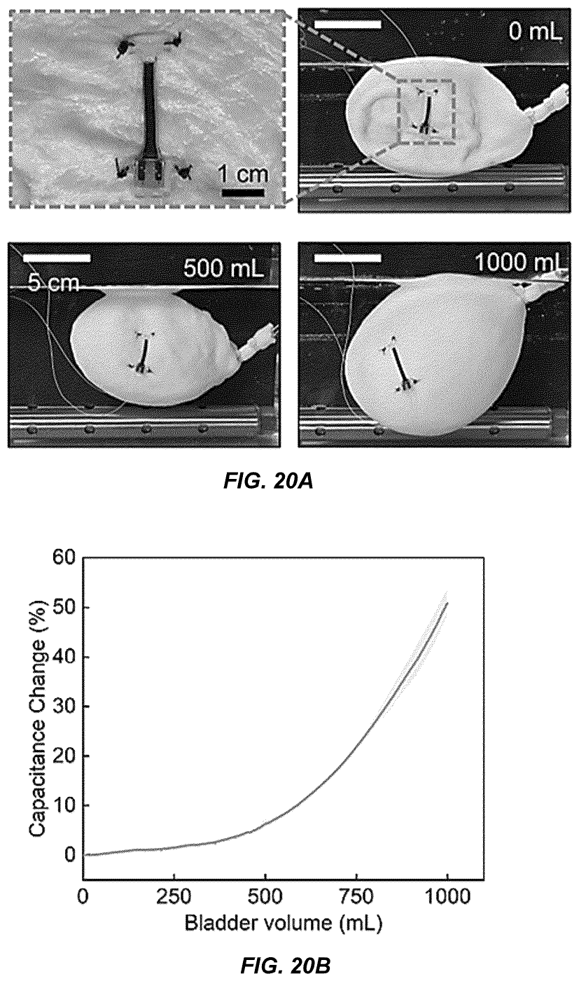

Ex vivo studies on a porcine bladder further validate of the example implantable strain sensor of this disclosure for bladder volume sensing. In this test, 8 markers were first sutured at the center of the bladder surface to measure the strain distribution during bladder filling. The axial and circumferential strain levels were 11.2% and 30.9%, respectively, after 500 mL injection. The strain then increased to 12.1% and 52.6% at the axial and circumferential direction, respectively, after 1000 mL injection. The strain was small at the initial stage of volume expansion probably because the bladder's luminal surface contains rugae and wrinkles that were flattened to accommodate expansion and tension without stretching. Since the bladder demonstrated larger strain at its circumferential direction, the example sensor (4 μm parylene, 120% pre-strain) was placed along this direction when the bladder is empty and sutured on the external tissue via 4 suture holes as shown in FIG. 20A. FIG. 20A specifically shows images of ex vivo porcine bladder with a sutured strain sensor (4 μm annealed parylene, 120% pre-strain) at different volumes: 0 mL, 500 mL, and 1000 mL volume. After immersing the bladder in 37° C. PBS, 1000 mL PBS was injected into the bladder at a rate of 160 mL/min.

FIG. 20B is a graph that shows sensor capacitance change as the bladder volume changes from 0 to 1000 mL. The lighter shaded area indicates the standard deviation from 3 trials. FIG. 20B shows the sensor's capacitance change over 3 cycles of injections, which has an average 6.3% change within 500 mL, and 50.7% change at 1000 mL. The capacitance change indicates the strain on the sensor is only about 2.2% and 25.4% at 500 mL and 1000 mL volume, respectively, which are lower compared to the surface strain without the sensor. Such constraint on bladder deformation is probably caused by the nonlinear “triphasic” response of stress-stretch relationship in bladder tissues, in which it is ultra compliant at small stretch and then shows linear elastic behavior at large stretch.

In another example, wrinkled parylene encapsulation can be achieved by first uniaxially pre-stretching a silicone-based strain sensor using a custom stretcher. After fixing both ends of the sensor at a certain level of prestrain, the stretcher can be placed in a parylene coater to uniformly deposit one or more layers of parylene C (e.g., having a thickness of approximately 1 to approximately 5 μm) on the surface of the pre-stretched sensor. In an example, the encapsulation material, e.g., parylene, is coated or deposited via a chemical vapor deposition process. Releasing the sensor's pre-strain after coating induces local buckling on the parylene C film and spontaneously forms wrinkles (e.g., the surface morphology of the film transforms from smooth to wavy). Connecting leads to the sensor and covering the connection with biocompatible epoxy finishes the sensor fabrication with a complete encapsulation. Stretching the sensor within the range of previously applied pre-strain simply transforms the wrinkled parylene to its original flat state without fracturing, thus preserving both large stretchability and encapsulation.

In some aspects, to demonstrate the stretchability of the wrinkled parylene film, it was applied on a parallel-plate stretchable capacitive strain sensor. In an example, the sensor surface was coated with approximately 3 μm of parylene C with 75% pre-strain creating a wrinkled composite on the sensor surface upon release of the pre-strain. Even though parylene's modulus is 3˜4 orders higher than that of elastomers, the composite formed can be stretched under at least 40% applied uniaxial strain without fracturing, and in an example the load only increases 75% after coating parylene. In addition, loading and unloading curves indicate the wrinkled parylene has little signal hysteresis in the sensor. Also, the time-dependent capacitance change at 30% strain was tested before and after applying the wrinkled parylene, showing only minimal drifting in the capacitance value.

In an example, aging tests were conducted in liquid environments to demonstrate the encapsulation capability of the wrinkled parylene film. A coated capacitive strain sensor was first immersed in phosphate buffered saline (PBS) at ambient temperature. The sensor remained unstretched in PBS for approximately 7 days and its capacitance value only had +/−2% changes. In an example, thermally accelerated aging tests were performed by bonding another coated capacitive strain sensor on an organ phantom and immersing it in PBS at 50° C. The sensor's capacitance increased as the phantom was inflated. Over a 7-day period, the phantom was repeatedly inflated and deflated, and the trends of capacitance change remained similar. These two tests demonstrated that the wrinkled parylene as provided herein could provide sufficient encapsulation for stretchable sensors.

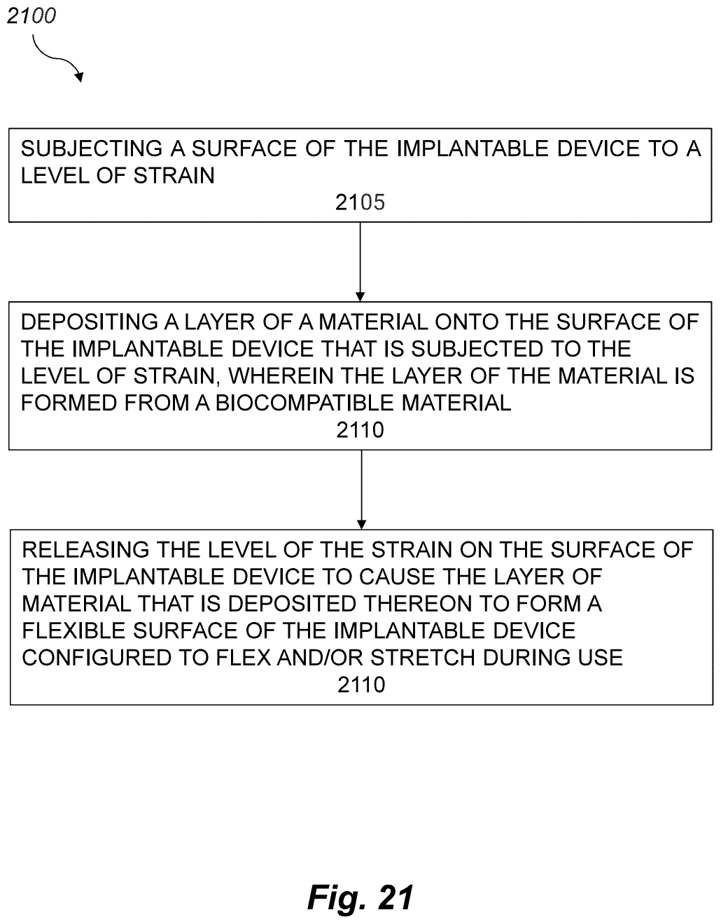

In some aspects, a method 2100 is disclosed in FIG. 21 for stretchable encapsulation on a stretchable surface of an implantable device. Step 2105 of method 2100 can include subjecting a surface of the implantable device to a level of strain. Step 2110 of method 2100 can include depositing a layer of a material onto the surface of the implantable device that is subjected to the level of strain, wherein the layer of the material is formed from a biocompatible material. Step 2115 can include releasing the level of the strain on the surface of the implantable device to cause the layer of material that is deposited thereon to form a flexible surface of the implantable device configured to flex and/or stretch during use.

Although systems and methods have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1A-21 may be modified, and that the foregoing discussion of certain of these embodiments does not necessarily represent a complete description of all possible embodiments. For example, one or more of the procedures, processes, or activities of systems and methods of this disclosure may include different procedures, processes, and/or activities and be performed by some different operation in some different order.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

The specific configurations, choice of materials, concentrations thereof, steps in preparing, and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. An implantable device configured to be used within a human body, comprising:
a plurality of electrodes positioned between at least one encapsulation layer and an outer film layer, the outer film layer configured to flex or stretch and comprising a biocompatible material.

2. The implantable device of claim 1, wherein the at least one encapsulation layer comprises a first elastomeric layer positioned on a first side of the plurality of electrodes and a second elastomeric layer positioned on a second side of the plurality of electrodes opposite the first side.

3. The implantable device of claim 1, wherein the at least one encapsulation layer comprises a first elastomeric layer positioned between electrode layers of the plurality of electrodes.

4. The implantable device of claim 1, further comprising a plurality of clincher tabs positioned on a first lateral end of the implantable device in communication with the plurality of electrodes.

5. The implantable device of claim 1, wherein a dielectric layer of the plurality of electrodes comprises a thickness between approximately 150 μm to approximately 200 μm.

6. The implantable device of claim 1, wherein the plurality of electrodes comprises a plurality of nanotube and an elastomer as a dielectric material.

7. The implantable device of claim 1, wherein the outer film layer comprises an inorganic material layer sandwiched between an outer layer and an inner layer each different from the inorganic material layer.

8. The implantable device of claim 7, wherein the outer layer and the inner layer comprise a material water vapor transmission rate (WVTR) lower than parylene.

9. The implantable device of claim 1, wherein the outer film layer comprises a wavy or wrinkled surface that comprises parylene.

10. The implantable device of claim 1, wherein the outer film layer is configured to be stretched to up to about 60-80% uniaxial strain without fracturing; and/or
wherein the implantable device comprises at least approximately 60% stretchability.

11. The implantable device of claim 1, wherein the implantable device comprises a doubled gauge factor.

12. A method of stretchable encapsulation on a stretchable surface of an implantable device, comprising:
subjecting a surface of the implantable device to a level of strain;
depositing a layer of a material onto the surface of the implantable device that is subjected to the level of strain, wherein the layer of the material is formed from a biocompatible material; and
releasing the level of the strain on the surface of the implantable device to cause the layer of material that is deposited thereon to form a flexible surface of the implantable device configured to flex and/or stretch during use.

13. The method of claim 12, wherein the subjecting the surface of the implantable device to the level of strain comprises uniaxially pre-stretching the implantable device to a pre-strain of between approximately 30% to approximately 150%.

14. The method of claim 12, wherein the depositing the layer of the material onto the surface of the implantable device that is subjected to the level of strain comprises conformally coating the layer comprising a thickness of between approximately 0.5 μm to approximately 10 μm, via chemical vapor deposition.

15. The method of claim 12, wherein the releasing the level of the strain on the surface of the implantable device to cause the layer of material that is deposited thereon to form the flexible surface comprises releasing a pre-strain of the implantable device to induce local buckling in the outer layer to form microscale wrinkle surfaces.

16. The method of claim 12, further comprising:
annealing the deposited layer beyond its glass transition temperature to increase crystallinity and/or form ordered and folded polymer crystallites; or
annealing the deposited layer beyond its glass transition temperature causing a strain range of the implantable device to increase at least 100%.

17. The method of claim 12, wherein the flexible surface comprises a wavy or wrinkled surface displaying over about 60% uniaxial stretchability.

18. The method of claim 12, wherein the layer of the material comprises parylene C.

19. The method of claim 12, wherein the step of depositing is formed by chemical vapor deposition.

20. The method of claim 12, wherein the implantable device is a soft strain sensor comprising at least approximately 60% stretchability.
